(19) United States
(12) Patent Application Publication
(10) Pub. No.: US 2012/0232554 A1
(43) Pub. Date: Sep. 13, 2012

(54) Alignment Plate for Lower-extremity Ring Fixation, Method of Use, and System

(75) Inventors: Alexander Shaevitz, Salem, OR (US); Doug Beaman, Hood River, OR (US); Randall J. Huebner, Portland, OR (US); Rachel Dreilinger, Lake Oswego, OR (US)

(73) Assignee: QUANTUM MEDICAL CONCEPTS LLC, Portland, OR (US)

(21) Appl. No.: 13/415,491

(22) Filed: Mar. 8, 2012

Related U.S. Application Data
(60) Provisional application No. 61/450,709, filed on Mar. 9, 2011.

(51) Int. Cl. A61B 17/62; A61B 17/60

(52) U.S. Cl. 606/56; 606/54

(57) ABSTRACT

An alignment plate having a viewing window and three adjustable strut members enables a surgeon to better place external fixation pins and an external fixation ring system relative to a desired anatomical feature of the lower extremity.

### PRIORITY CLAIM

The present application claims benefit under 35 USC Section 119(e) of U.S. Provisional Patent Application Ser. No. 61/450,709 filed on 9 Mar. 2011. The present application is based on and claims priority from this application, the disclosure of which is hereby expressly incorporated herein by reference.

### BACKGROUND

The present invention relates to therapeutic orthopedic devices for fixating, immobilizing, and manipulating the human anatomical skeleton, particularly the foot and lower leg. And, more specifically, the present invention relates to devices that aid initial location (pre-operative and operative) of external (ring) frame systems for particular use for fixating lower leg bones and the foot with respect to the tibia.

External Fixation devices locate segments of bone relative to a reference location of the skeleton for varied purposes including reconstruction of fractured or deformed extremities. By distracting or compressing portions of the anatomical skeleton, external fixation devices can correct angulation, rotation, and translation of targeted bones or bone segments.

External fixation—an orthopedic procedure utilizing external fixation devices—involves surgically securing bone pins both above and below a bone fracture or chosen site for manipulation, providing attachment points that may couple with or to another component, such as a clamp or frame member, of the external fixation system.

One particular form of external fixation, circular fixation (also called external ring fixation), is a proven medical treatment technique to overcome, correct, or repair many problems of the lower leg and foot stemming from traumatic injuries, infections, non-unions, or congenital abnormalities.

External fixation can practically manipulate bone in any desired combination of translation, rotation, angle, or length. When applied to the foot and lower leg, external fixation treatments include the use of structures incorporating one or more external ring frame members (semi-circumferential or full circumferential frame members) to encompass and stabilize the limb by pins and or tensioned wires. The frames are employed to capitalize on these biologic phenomenae that govern tissue (in particular, bone) growth under tension and optimized distraction rates.

Bone regrowth as a result of circular fixation applies an accepted treatment termed controlled distraction histogenesis, whereby bone is fractured and then slowly lengthened at a very specific and controlled rate to optimize bone regrowth. As the fractured bone is distracted, new bone growth occurs in the fracture region and establishes a new segment of healthy bone in the defect. The tension that is created by gradual distraction stimulates the formation of new bone, skin, blood vessels, peripheral nerves, and muscle. Circular fixation thus allows for an external means of manipulating translational, rotational, angular, and even length discrepancies while preserving soft tissue from excessive trauma that would otherwise prevent early motion and use of the limb.

Yet, to obtain the maximum therapeutic benefit of external ring fixation, a skilled surgeon must precisely locate the external frame members, which carry and position the pins that penetrate the skeletal frame, aligning the various pins and external frames with abstract reference planes based on the position of the tibia or other major structure. This process of locating the external frames not only requires skill of the surgeon, it is a very time-consuming task and is fraught with the potential for errors. There is no existing mechanical alignment device that can assist the surgeon in placing the external frames and, as such, only time-honed skill, luck, and persistence during surgery provides any meaningful results.

Thus, there is a need for an external alignment guide device, method, and system that greatly reduces the guesswork and skill required to precisely and efficiently place external frames.

### DRAWING

FIG. 1 is an offset frontal view of a preferred embodiment of an alignment plate according to the present invention.

FIG. 2 is an offset frontal view of an aligning bracket according to a second preferred embodiment of the present invention.

FIG. 3 is an offset frontal view of the embodiment of FIG. 1 further including a drill guide.

FIG. 4 shows the embodiment of FIG. 1 relative to a typical external ring fixations system.

FIG. 5 shows the embodiment of FIG. 2 in a typical environment of use.

FIG. 6 is a front view of a drill guide according to a preferred embodiment of the present invention.

FIG. 7 is an exploded view of the drill guide of FIG. 6.

FIG. 8 is an end view of a stylus of the embodiment of FIG. 6.

FIG. 9 is a front view of the stylus of FIG. 8.

FIG. 10 is a front view of a mounting bracket component of the drill guide of FIG. 6.

FIG. 11 is an offset view of a component of the system according to a preferred embodiment of the present invention.

FIG. 12 shows an additional component added to the system depicted in FIG. 11.

### DESCRIPTION OF THE INVENTION

Possible embodiments will now be described with reference to the drawings and those skilled in the art will understand that alternative configurations and combinations of components may be substituted without subtracting from the invention. Also, in some figures certain components are omitted to more clearly illustrate the invention.

As will become apparent to those of ordinary skill in the art, the teachings of the present invention may be employed in many different constructs for external fixation depending on the particular surgical application and further depending on surgeon preferences. The construct shown in the drawings are merely exemplary. The various components of the system of the present invention may be alternatively utilized in different constructs involving some or all of the illustrated components. Additionally, the various components of the present invention may be used in connection with other components, some are commercially available, as would be appreciated by those of ordinary skill in this art.

The use of external fixation frames for aligning and retaining broken or fractured bones in a particularly desired orientation or configuration is widely known and commonly employed. However, in most typical external frame constructions, particularly frame systems adapted to align bones of the lower extremity, a variety of clamps, rings, rods, and holding structures enable the skilled surgeon to position the broken or fractured bones in precise location, position and orientation (typically relative to the tibia), and then allowing the bone to fixed in that position for complete healing.

Although a wide variety of frame structures and clamp systems are widely available for this purpose, a common problem is the requisite skill level of the surgeon. Highly skilled surgeons can more quickly and efficiently align existing systems, but newer surgeons require more time. Yet, even the most experienced surgeon still has difficulty with precise positioning of the external frame elements relative to the bone. This difficulty translates into additional time that the patient must be sedated, which in turn increases risk of harm to the patient. Further, improper alignment of the external frame components is common and may lead to inaccurate corrections of the extremity, improper frame construction of the external fixation elements that limits proper weight bearing resulting in improper healing or additional post-operative treatment time, healing time, or supplementary care.

The current art instructs inserting an external anchor pin or screw into a fragmented or broken or misaligned bone at one end of the pin or anchor and fixing the opposite end of the external anchor rod, anchor pin or anchor screw to a ring fixation member. The fragmented, misaligned, or broken bone segment is aligned, in part, during surgery, and its relative location is anchored by means of the external rod, pin, or screw via the external frame member, which in turn, is fixed relative to the tibia or other skeletal support selected by the surgeon. Then, smaller adjustments to precisely position the fragment may be done post operatively, as required.

The difficulty with the known art lies in establishing a reference plane for locating the external ring fixation members relative to an internal skeletal component (such as the tibia or the bottom plane of the foot). Until the present invention, the surgeon estimated, guessed, or “eye-balled” the desired position of the various rings and relied solely on experience and trial-and-error to get the position correct while the patient is sedated. It should be appreciated that surgeon's skill levels vary, and the lack of any solid reference plane led to guess work, which in turn led to mistakes. If these mistakes were detected during surgery, the patient would continue to be sedated and the error corrected, perhaps without the patient ever knowing—yet being exposed to increased risk as the length of the sedation increased, and increased exposure to foreign contaminants as the operative site was left open for longer durations.

Recognizing these hazards, more recent attempts to provide improved systems for external fixation utilized more specialized clamp systems to enable more rapid and repeatable adjustments. For example, U.S. Pat. No. 6,613,049 issued to Wainquist et al. on 2 Sep. 2003 describes an adjustable bone stabilizing frame system. Wainquist instructs that a clamping member having an internally mounted friction pin enables quick and easy construction of a basic frame structure. While Wainquist's system enables quick adjustment of the basic frame components, it fails to address a fundamental problem: aligning the frame components in the first instance to a particular skeletal member, or alternative, to an external feature that directly correlates to known structures and desired alignments of the fractured bones to properly heal.

Other external fixation devices for the repair of the anatomical skeleton are known. One state-of-the-art representative device, hereby incorporated by reference as if fully set forth herein, includes the external fixation system described by Walulik et al. in U.S. Pat. No. 6,277,119 issued on 21 Aug. 2001. The Walulik device includes interchangeable and distinct components that allow for a greater degree of surgeon flexibility in producing a desired construction to secure bone portions with bone pins. These components include at least one cylindrical support rod and a plurality of universal clamp assemblies for engagement with at least one support rod.

Specialized external fixation systems that enable a patient to bear weight on the fixed lower extremity are also generally known. Such specialized systems enable the gradual increase of the patient's own weight on the immobilized limb to hasten recovery and promote tissue and bone regeneration. One representative example of such specialized lower-extremity external fixation systems include the combination bone fixation/immobilization apparatus of Grant et al., described in U.S. Pat. No. 6,964,663 issued on 15 Nov. 2005 and this disclosure is hereby incorporated by reference as if fully set forth herein. The Grant device includes a walking attachment adapted to have a plurality of transfixation wires fixed thereto and includes a substantially rigid leg support assembly comprising a cuff and strap.

Another external fixation system, described by Ferrante et al. in U.S. Pat. No. 7,048,735 issued on 23 May 2006, includes clamping elements that provide three-axes of rotation relative to the other capture member.

Problems common to the current-state-of-the-art devices, represented above, include an unacceptable level of precision that must be maintained during the creation of the frame structure in orientating each component while a multitude of fasteners are tightened. Further, as many of the components interact with other components, manipulation or adjustment of one selected clamp, for instance, requires cooperating adjustments to several sub-systems and fasteners. Not only are such adjustments time-consuming, they are often impossible for all but the most skilled surgeons to properly locate relative to a desired reference plane.

To overcome these known problems and limitations and to reduce the requisite skill, eliminate guess-work, and otherwise make more efficient and reduce patient-risk, the present invention presents a system and method for aligning most any external ring fixation devices. Although the preferred embodiments described herein specifically contemplate the lower extremity of human patients, the concepts, specific embodiments, elements, and components of the present invention can be readily adapted by those skilled in the art for aligning other anatomical structures.

### Alignment Plate

A first preferred embodiment of the present invention contemplates an alignment plate 10 for aligning an external frame fixation system. The alignment plate comprises a plate body 16 comprising a substantially planar surface having first flange 18 comprising a first-flange leg 17 extending downward from a top surface of the plate body on a first side of the top surface of the plate body, the first flange then extending away from the first-flange leg providing a substantial planar first flange surface 15 that is substantially parallel to the top surface.

The plate body further includes a second flange 19 comprising a second-flange leg 23 extending downward from the top surface on a second side, the second side being opposite the first side, the second flange 19 then extending away from the second-flange leg providing a substantial planar second flange surface 21 that is substantially parallel to the top surface and substantially co-planar to the first flange.

The plate body 16 further comprising a handle portion 20 disposed in the top surface.

The plate body further comprising a window 14 aperture arranged in the flat surface, the window aperture comprising a transparent material adapted to couple to the plate body and adapted to provide a window top surface that is substantially flat and coplanar to the top surface of the plate body. One contemplated transparent material well suited as a window aperture 14 includes materials such as polycarbonate or polysulphone or other similar materials as would be understood in the art.

The alignment plate further comprising a heel-stop plate 12 adjustably disposed on the top surface of the plate body.

The first flange 18 and second flange 19 further each respectively including a front strut support slot 25.

The alignment plate 16 further comprising a first-flange (front) strut 24 adapted to selectively position in a first front flange support slot 25 and a second-flange strut 26 adapted to selectively position in a second flange front support slot 25a, and a heel strut (rear strut) 22 coupled to the top surface of the plate body adjacent to the heel-stop plate 12. And, each respective strut comprises a threaded rod portion adjacent to a distal end of the strut, a spring member overlaying the threaded rod portion, a spring stop adjacent to the spring member and overlaying an intermediate portion of the strut, the spring stop further engaging a portion of the flange or top surface, each strut further comprising a proximal adjusting end adapted to rotate, thus causing the threaded portion to displace along its long axis.

The alignment plate 16 further comprising a pair of front slots 28 adapted to receive a front-foot strap (not illustrated in the figures of the drawing) and a pair of rear slots 30 adapted to receive a rear-foot strap (not illustrated).

### Drill Guide

The system 10 of the present invention further contemplates a drill guide 40 adapted to mount the plate body 16 and/or flange 18 or 19. The drill guide 40 comprises a mounting strut 41 adapted to selectively couple to the plate body and or either flange of the plate body. The strut includes a mounting feature consisting of a horizontal slot 58 arranged on a lower portion of the strut 41 and adapted to slide over a portion of the plate body and or either flange. The mounting strut selectively couples or tightens against the plate body by means of a tightening assembly 56, which consists of a threaded rod adapted to engage a corresponding threaded hole on the strut and a thumbwheel to make the tightening or loosening of the tightening assembly. The strut further includes a vertically arranged, elongated slot 43 extending from a portion of the strut adjacent to the mounting feature and extending upward to the distal end of the strut.

The drill guide 40 further includes a mounting fixture 42 adapted to hold a stylus 44 and pointing guide extension arm 52. The mounting fixture consists of a fixture body 51 having a threaded hole 53. A threaded rod with a thumbscrew 55 on one end selectively inserts in the threaded hole on the fixture body. Thus, when positioned relative to the vertical slot 43 of the strut 41, the mounting fixture can be selectively positioned along the vertical axis defined by the strut and can further rotate 360-degrees around an axis defined by the threaded rod. Accordingly, the stylus 44 can be positioned to point at any desired target on the lower extremity.

The mounting fixture further has a top slot 57 disposed on a top portion to form a pointing guide holder channel for the pointing guide.

The drill guide 40 further comprises a stylus body 44 having a guide aperture 46 at a first end and an oppositely disposed guide-pointing end 50. The two ends defining an elongated cylindrical body having a hollow cylindrical channel 48 (not labeled in the drawings) adapted to receive an external fixation rod, screw, or wire. The stylus body 44 adapted to slideably engage a corresponding the stylus holder channel 46 in the body 51.

And, the drill guide 40 further comprising a guide extension arm 52 having a first end mounted in a mounting base 60. The mounting base adapted to slideably engage the slot 57 on the mounting fixture. The guide extension arm further having a second end comprising a downward pointing guide extension distal pointing finger 54.

### Aligning Bracket

In a second preferred embodiment, the system of the present invention includes an aligning brace 100 consisting of a generally U- or C-shaped washer clip. The aligning brace 100 for an external frame fixation system comprises a body member 110 comprising a resiliently deformable material. The clip body has a general C-shape with a first clamp hand 120 at a first end and a second clamp hand 130 at an opposite second end, the first and second clamp hands arranging to be adjacent to each other. Further, each respective first and second clamp hand further comprises first lip 152 and a second lip (152), the first and second lip cooperating to define a channel 154.

The aligning brace 100 further comprises a first spherical washer assembly 160 (not illustrated in the figures of the drawing), such as a spherical washer assembly is well known by those skilled in this art and is widely available. Moreover, the washer assembly 160 adapts to couple to the channel of the first clamp hand. Similarly, a second spherical washer assembly (not separately numbered in the drawing) adapts to couple to the channel of the second clamp hand. As is understood by those skilled in this art, each respective spherical washer assembly comprises a cup having a rib and a pivot washer having a raceway, the raceway adapted to engage the cup.

### Method of Use

Alignment plate 10, while held in surgeon's hand, is pressed against the patient's foot so that the heel is in firm contact with the heel plate 12, or optionally, the planar surface of the foot without use of the heel plate. A ring fixation element 11 is held in an estimated position offset from the heel plate to determine a rough offset of the ring from the alignment plate. Next, the surgeon disengages the patient's foot with the alignment plate. The ring element is coupled to the alignment plate 10 by means of the rear 22, first front 24 and second front 26 strut assemblies. Once again, the surgeon places the alignment plate against the patient's foot as described, above. Precise positioning of the ring relative to the alignment plate while the alignment plate is against the sole of the foot (planar surface) is accomplished by turning the handle end of each respective strut.

The drill guide 40 is coupled to the ring alignment plate and positioned over the patient's foot in an orientation decided by the surgeon for external pin fixation. Additional adjustments to the ring, by adjusting the struts (turning their respective handles) can position the alignment plate relative to an appropriate anatomical feature of the lower extremity. The surgeon may look through the window 14 to better view the position of the patient's foot relative to the alignment plate and make adjustments to the struts as needed.

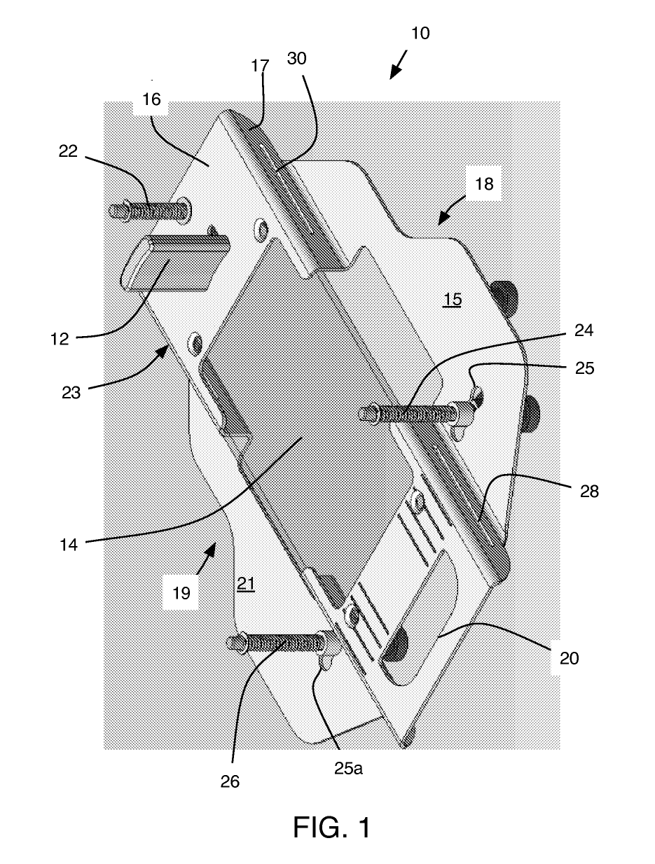

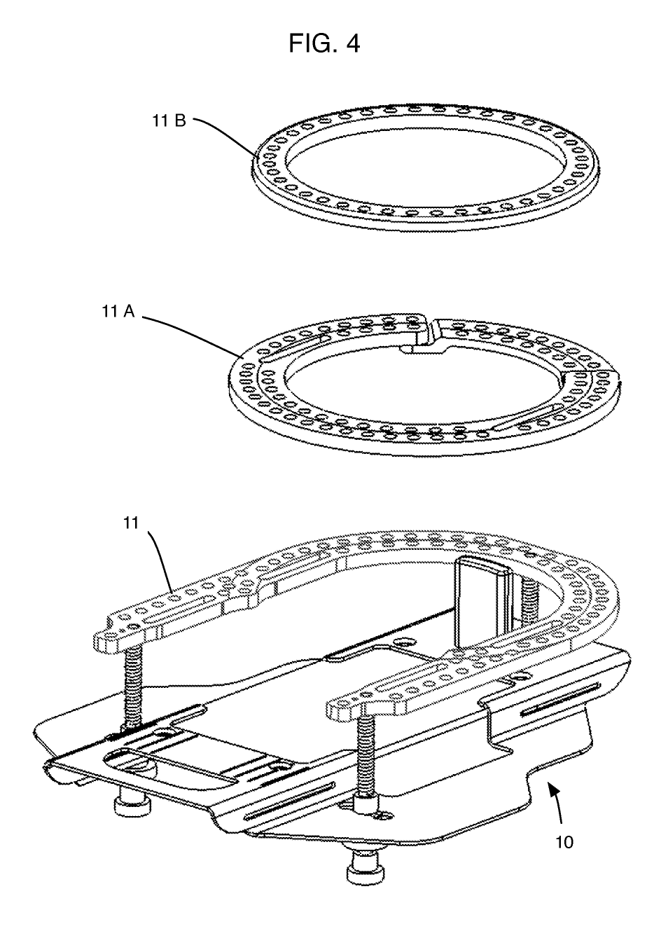

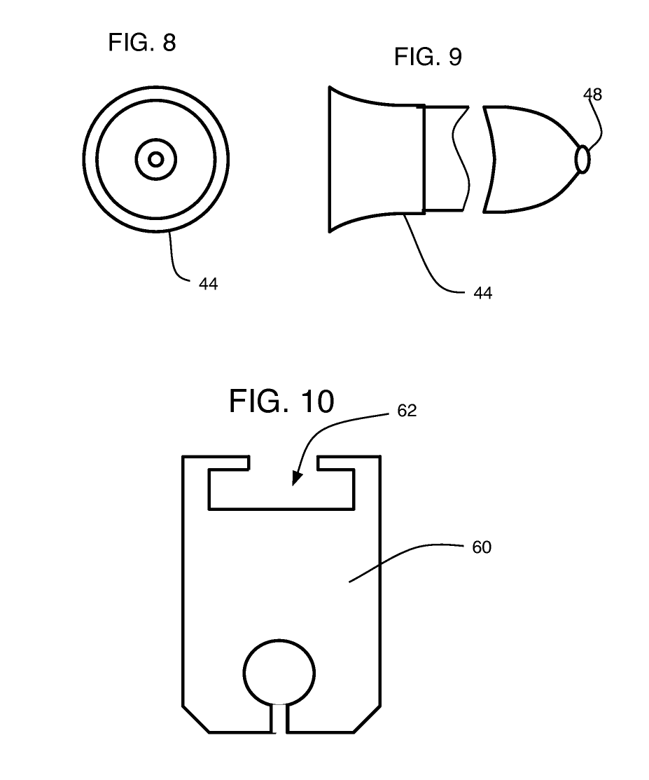

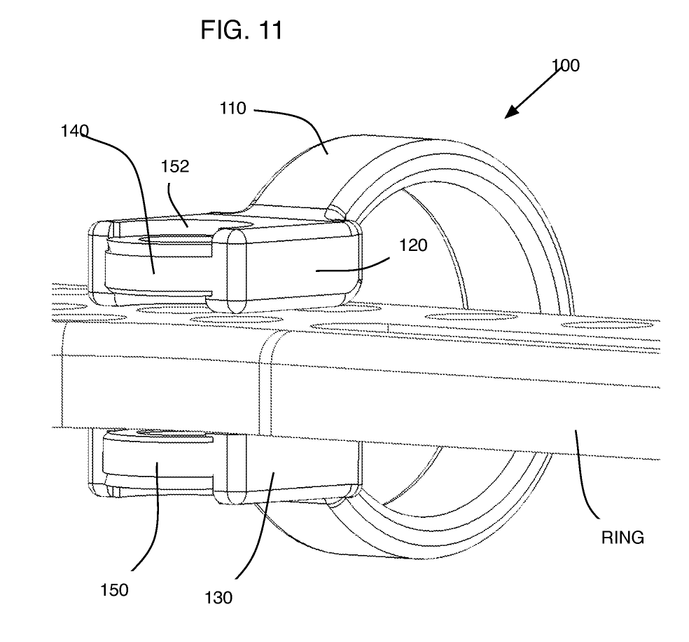

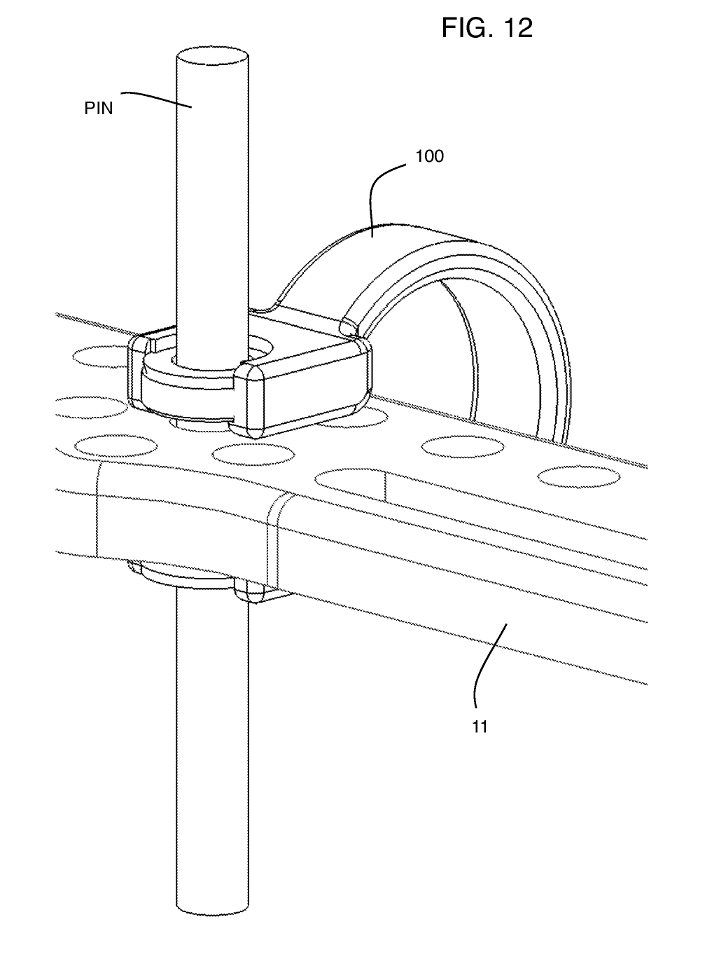

FIGS. 1-12 illustrate a preferred embodiment and alternate embodiments of the system and devices of the present invention. Accordingly, as FIG. 1 illustrates in part, the present invention includes an alignment system 10 adapted to use with a lower extremity external frame fixation system to position relative to anatomical features of the lower extremity. The alignment system comprises a plate body 16 having a substantially planar top surface. This surface is adapted to be placed against a planar surface of a foot of the lower extremity.

The plate body creates a first plane with its top planar surface. This establishes a reference plane. Extending downward from the plate body a pair of wings or flanges that arrange parallel to, but offset from, this reference plane. A first flange 18 has a first-flange leg 17 extending downward from the top surface of the plate body on a first side of the top surface of the plate body, the first flange then extending away from the first-flange leg providing a substantial planar first flange surface 15 that is substantially parallel to the top surface of the plate body.

A second flange 19 comprising a second-flange leg (similar to the first flange leg, but FIG. 1 does not show this leg due to the viewing perspective) extends downward from the top surface on a second side, the second side being opposite the first side, the second flange then extending away from the second-flange leg providing a substantial planar second flange surface 21 that is substantially parallel to the top surface and substantially co-planar to the first flange surface.

The plate body further includes an opening or window aperture 14 disposed on the top surface of the plate body. This opening is preferably covered by a translucent sheet of glass or plastic or plexiglass or other similar material as would be understood in the art, to enable a surgeon to look through the plate when placed against the planar surface of the patient's foot to assist in aligning the external fixation system relative to the plate and anatomy of the patient. Accordingly, this window aperture 14 is a transparent material adapted to couple to the plate body and adapted to provide a window top surface that is substantially flat and coplanar to the top surface of the plate body.

Optionally and as FIG. 1 shows, the system 10 contemplates the alignment plate 16 including a heel-stop 12 adjustably disposed on the top surface of the plate body. The heel-stop acts as a point of reference for assisting alignment of the foot relative to the plate body and external fixation system. A patient's heel can be placed against the heel stop. Because surgeons may desire different fitments of an external fixation system, the heel stop is adjustable fore and aft.

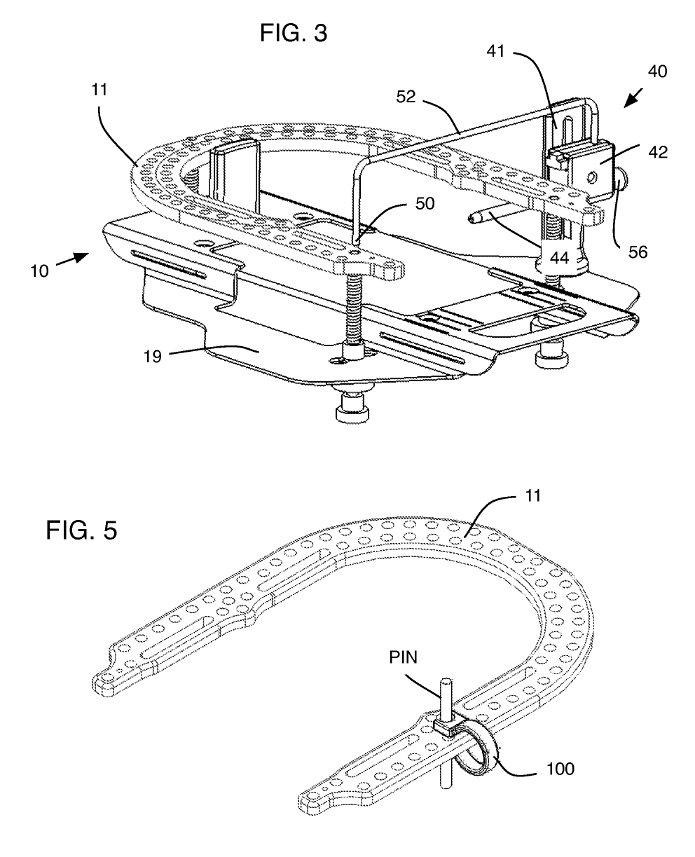

Extending upward from the reference plane created by the plate body and adapted to couple to an eternal ring component of an external ring fixation system, the plate body includes three struts 22 24 26. Specifically, a first strut 24 adapted to selectively couple to a corresponding first slot 25 arranged on the plate body, a second strut 26 adapted to selectively couple to a corresponding second slot 25a arranged on the plate body, and a third strut 22 adapted to selectively couple to a corresponding third slot (not enumerated in the drawing) arranged on the plate body provide three points to attach an external ring (see FIG. 3, for example). Each strut includes a mechanism such as a threaded portion at its proximal end and nut or pair of nuts to selectively couple to the plate and as such can be adjusted individually for vertical height relative to the top surface (reference plane) of the plate body. At its distal end, each strut includes a landing feature for a corresponding mounting hole provided by the external ring. This landing feature looks like a flat washer mounted to a portion of the strut near the terminus of its distal end (see, e.g., FIG. 1), and prevents the plate (see, e.g. FIG. 3) from sliding down the strut.

In another contemplated preferred embodiment, the system as just described above cooperates with external ring fixation systems. Such external ring fixation systems include an external ring fixation element 11 (see, e.g., FIGS. 3, 4, and 5) adapted to selectively couple to at least three struts, each strut having a first end adapted to couple to the plate body and a second end adapted to selectively engage the ring fixation element, each strut further being adapted to adjust a relative vertical offset of the ring fixation element relative to the plate body.

Contemplated external ring fixation elements 11 include a U-shaped ring element, or an oval or round 11A or series of ring elements 11 11A 11B or any combination of ring elements.

Although not shown in the figures of the drawing, the preferred embodiments of the present invention cooperate with existing guide wires and such, as would be well understood and appreciated by those skilled in this art. Accordingly, the alignment system 10 includes a wire adapted to couple to the ring element and further adapted to insert into an anatomical feature of the lower extremity.

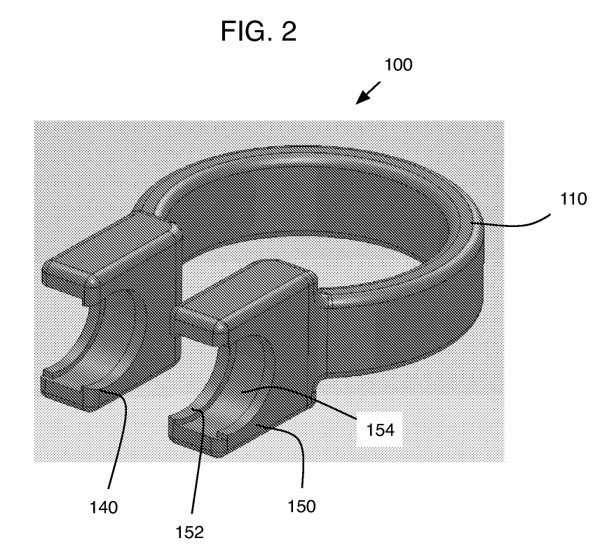

As FIGS. 2, 11 and 12 show, the system 10 can further include an aligning brace 100 adapted to selectively insert relative to the external frame fixation system. The aligning brace 100 includes a body member 110 constructed of a resiliently deformable clip body having a general C-shape, U-shape, near oval shape and the like. The body member having a first clamp hand 140 at a first end and a second clamp hand 150 at an opposite second end. The first and second clamp hands arranging to be adjacent to each other by being affixed to opposite ends of the curving body 110. And, each respective first and second clamp hand further comprises first lip 152 and a second lip, the first and second lip cooperating to define a channel.

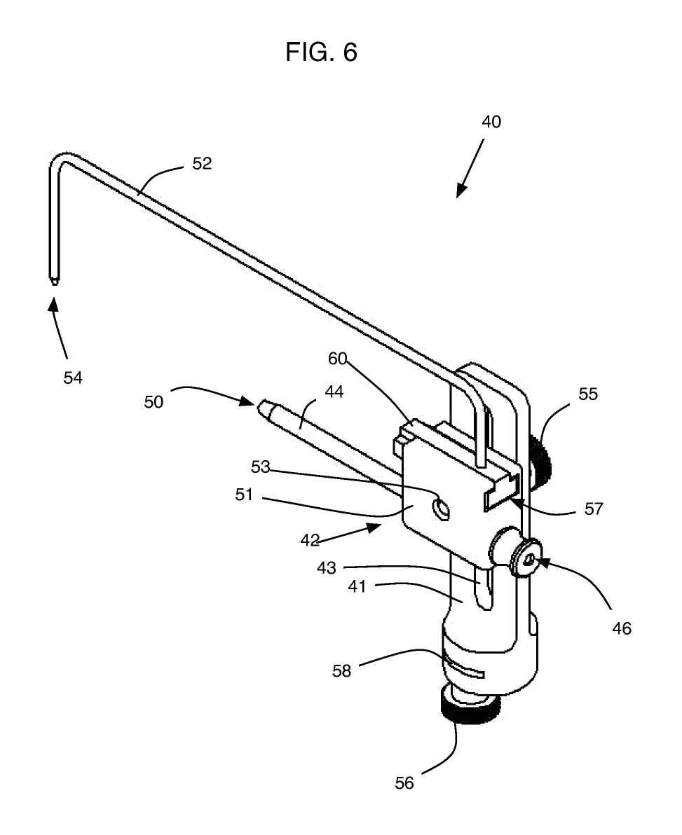

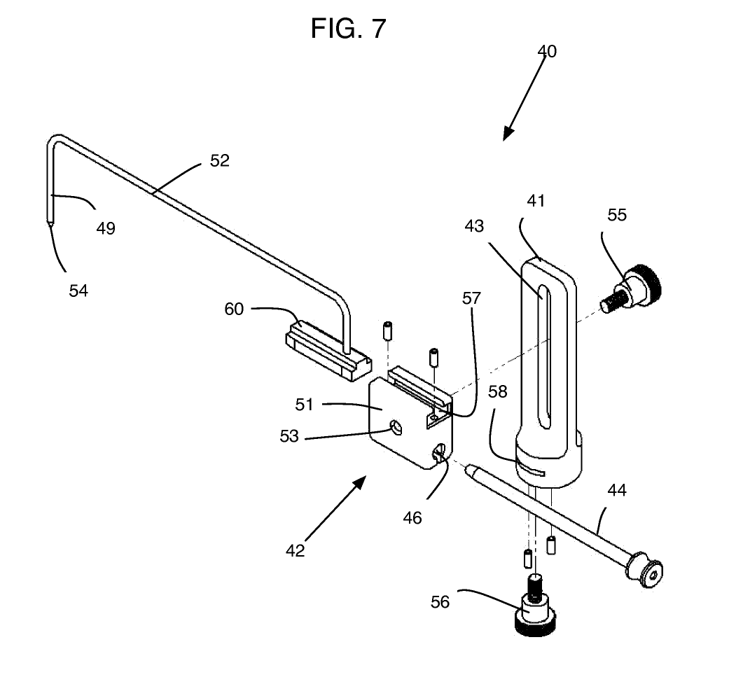

FIG. 3 shows the system 10 including a drill guide 40 (detailed in FIGS. 6 and 7, for example). The drill guide 40 includes a mounting strut 41 adapted to selectively couple to the plate body. The strut comprises a mounting feature comprising a horizontal slot 58 arranged on a lower portion of the strut 41, the horizontal slot being adapted to slide over a portion of the plate body and or either flange. The strut 41 further includes a vertically arranged, elongated slot 43 extending from a portion of the strut adjacent to the mounting feature 58 and extending upward to the distal end of the strut. The strut 41 couples to a mounting fixture 42 adapted to hold a stylus 44 and pointing guide extension arm 52. The mounting fixture comprises a fixture body 51 having a threaded hole 53 and a threaded rod with a thumbscrew on one end 55 that selectively inserts in the threaded hole on the fixture body whereby when positioned relative to the vertical slot of the strut the mounting fixture can be selectively positioned along the vertical axis defined by the strut and can further rotate 360-degrees around an axis defined by the threaded rod.

And, the mounting body 51 further holds a guide extension arm 52 having a first end mounted in a mounting base 60. The mounting base is configured to slideably engage and couple to a corresponding top-slot 57 on the mounting fixture. The guide extension arm 52 further has a second end 54 comprising a downward pointing guide extension distal pointing finger 49.

A contemplated preferred method of the present invention includes a method adapted for aligning an external fixation system relative to anatomical features of a lower extremity. This method comprises the steps of providing an alignment system comprising a plate body comprising a substantially planar top surface adapted to be placed against a planar surface of a foot; providing an element of an external ring fixation system; pressing the plate body against a planar surface of the lower extremity; estimating the offset of the ring element relative to the plate body; positioning roughly the ring element to the plate body and coupling the plate body to the ring element by means of one or more struts provided by the plate body; and positioning precisely the ring element relative to the plate body while the plate body is pressed against the planar surface of the foot.

This method further contemplates additional steps including providing a drill guide; aligning the drill guide on the plate body relative to the ring element and a desired anatomical feature.

Although the invention has been particularly shown and described with reference to certain embodiments, it will be understood by those skilled in the art that various changes in form and detail may be made without departing from the spirit and scope of the invention. And, although claims are not required, we claim at least:

We claim:

1. An alignment system adapted to use with a lower extremity external frame fixation system to position relative to anatomical features of the lower extremity, the alignment system comprising:
a plate body comprising
a substantially planar top surface adapted to be placed against a planar surface of a foot of the lower extremity, the planar top surface adapted to selectively couple to the lower extremity external frame fixation system.

2. The plate body of claim 1 further comprising:
a first flange comprising a first-flange leg extending downward from the top surface of the plate body on a first side of the top surface of the plate body, the first flange then extending away from the first-flange leg providing a substantial planar first flange surface that is substantially parallel to the top surface,
a second flange comprising a second-flange leg extending downward from the top surface on a second side, the second side being opposite the first side, the second flange then extending away from the second-flange leg providing a substantial planar second flange surface that is substantially parallel to the top surface and substantially co-planar to the first flange surface.

3. The alignment system of claim 1 wherein the plate body further comprises:
a window aperture disposed on the top surface of the plate body.

4. The window aperture of claim 3 further comprising:
a transparent material adapted to couple to the plate body and adapted to provide a window top surface that is substantially flat and coplanar to the top surface of the plate body.

5. The alignment plate of the alignment system of claim 1 further comprising:
a heel-stop adjustably disposed on the top surface of the plate body.

6. The alignment system of claim 1 further comprising:
a first strut adapted to selectively couple to a corresponding first slot arranged on the plate body;
a second strut adapted to selectively couple to a corresponding second slot arranged on the plate body; and
a third strut adapted to selectively couple to a corresponding third slot arranged on the plate body.

7. The alignment system of claim 1 further comprising:
an external ring fixation element adapted to selectively couple to at least three struts, each strut having a first end adapted to couple to the plate body and a second end adapted to selectively engage the ring fixation element, each strut further being adapted to adjust a relative vertical offset of the ring fixation element relative to the plate body.

8. The alignment system of claim 7 wherein the external ring fixation element comprises:
a U-shaped ring element.

9. The alignment system of claim 7 wherein the external ring fixation element comprises:
a oval-shaped ring element.

10. The alignment system of claim 7 wherein the external ring fixation element comprises:
a round-shaped ring element.

11. The alignment system of claim 7 further comprising:
a wire adapted to couple to the ring element and further adapted to insert into an anatomical feature of the lower extremity.

12. The alignment system of claim 1 further comprising:
an aligning brace adapted to selectively insert relative to the external frame fixation system, the aligning brace comprising a body member comprising a resiliently deformable clip body having a general C-shape, the body member having a first clamp hand at a first end and a second clamp hand at an opposite second end, the first and second clamp hands arranging to be adjacent to each other; and
each respective first and second clamp hand further comprises first lip and a second lip, the first and second lip cooperating to define a channel.

13. The alignment system of claim 1 further comprising: a drill guide comprising
a mounting strut adapted to selectively couple to the plate body, the strut comprises a mounting feature comprising a horizontal slot arranged on a lower portion of the strut, the horizontal slot being adapted to slide over a portion of the plate body and or either flange;
a vertically arranged, elongated slot extending from a portion of the strut adjacent to the mounting feature and extending upward to the distal end of the strut;
a mounting fixture adapted to hold a stylus and pointing guide extension arm, the mounting fixture comprises a fixture body having a threaded hole and a threaded rod with a thumbscrew on one end selectively inserts in the threaded hole on the fixture body whereby when positioned relative to the vertical slot of the strut the mounting fixture can be selectively positioned along the vertical axis defined by the strut and can further rotate 360-degrees around an axis defined by the threaded rod; and
a guide extension arm having a first end mounted in a mounting base, the mounting base adapted to slideably engage a corresponding top-slot on the mounting fixture, the guide extension arm further having a second end comprising a downward pointing guide extension distal pointing finger.

14. A method adapted for aligning an external fixation system relative to anatomical features of a lower extremity, the method comprising:
providing an alignment system comprising a plate body comprising a substantially planar top surface adapted to be placed against a planar surface of a foot;
providing an element of an external ring fixation system;
pressing the plate body against a planar surface of the lower extremity;
estimating the offset of the ring element relative to the plate body; and
positioning roughly the ring element to the plate body and coupling the plate body to the ring element by means of one or more struts provided by the plate body.

15. The method of claim 14 further comprising:
providing a drill guide;
aligning the drill guide on the plate body relative to the ring element and a desired anatomical feature.
